(19) United States
(12) Patent Application Publication
(10) Pub. No.: US 2026/0069201 A1
(43) Pub. Date: Mar. 12, 2026

(54) APPARATUS AND METHOD FOR MUSCULOSKELETAL EVALUATION USING IMAGES AND ALGORITHMS

(71) Applicant: SEOUL NATIONAL UNIVERSITY HOSPITAL, Seoul (KR)

(72) Inventors: Keewon KIM, Gwacheon-si (KR); Jinkyu LEE, Seoul (KR); Woosup CHO, Seoul (KR); Sun Hyung KIM, Seoul (KR)

(73) Assignee: SEOUL NATIONAL UNIVERSITY HOSPITAL, Seoul (KR)

(21) Appl. No.: 19/326,262

(22) Filed: Sep. 11, 2025

(30) Foreign Application Priority Data
Sep. 11, 2024 (KR) 10-2024-0124334

(51) Int. Cl. A61B 5/00; A61B 5/11

(52) U.S. Cl. CPC A61B 5/4519; A61B 5/0077; A61B 5/1116; A61B 5/7264

(57) ABSTRACT

Provided are a musculoskeletal evaluation apparatus using an image and an algorithm and a method thereof. The apparatus includes a memory that stores at least one process for performing musculoskeletal evaluation by using an image and an algorithm, a processor that performs an operation according to the process, obtains an image captured by a camera, calculates an evaluation index for evaluating at least one of a spine and a joint of a subject based on body coordinate information obtained from the image, and determines a state of at least one of the spine and the joint based on the calculated evaluation index.

### CROSS-REFERENCE TO RELATED APPLICATIONS

A claim for priority under 35 U.S.C. § 119 is made to Korean Patent Application No. 10-2024-0124334 filed on Sep. 11, 2024 in the Korean Intellectual Property Office, the entire contents of which are hereby incorporated by reference.

### TECHNICAL FIELD

The present disclosure relates to a musculoskeletal evaluation apparatus using an image and an algorithm, and a method thereof.

### BACKGROUND TECHNOLOGY OF INVENTION

Musculoskeletal disorders are musculoskeletal pains or damages to muscles, nerves, tendons, ligaments, bones, and surrounding tissues. They may be mainly aging-related changes, and may be aggravated by repetitive motion or pressure, stimuli such as vibration, improper working posture, and excessive force. They may be observed in all joints of the body, such as the neck, waist, shoulders, knees, arms, and legs, but are characterized depending on the joints.

Until now, to examine the health state of a musculoskeletal system, a person needs to visit a laboratory equipped with certain facilities and is evaluated under the assistance of an examiner.

However, the examining method involves the subjectivity of specialized personnel analyzing data, and is performed in specific spaces and locations, making it difficult to access the examination. Accordingly, it is difficult to identify a disease through screening tests in the early stages or conduct periodic follow-up observations. In addition, it is impossible to evaluate the disease because it does not reflect movements or postures in daily life.

### CONTENTS OF INVENTION

### Problem to be Solved

Embodiments of the present disclosure provide a musculoskeletal evaluation apparatus using an image and an algorithm, and a method thereof.

Problems to be solved by the present disclosure are not limited to the problems mentioned above, and other problems not mentioned will be apparent by those skilled in the art from the following description.

### Means of Solving Problem

According to an aspect of the present disclosure for achieving the above-described technical task, a musculoskeletal evaluation apparatus using an image and an algorithm includes a memory that stores at least one process for performing musculoskeletal evaluation by using an image and an algorithm, a processor that performs an operation according to the process. The processor obtains an image captured by a camera, calculates an evaluation index for evaluating at least one of a spine and a joint of a subject based on body coordinate information obtained from the image, and determines a state of at least one of the spine and the joint based on the calculated evaluation index. The image includes a first image, which is obtained when an evaluation mode is a routine activity mode, and a second image obtained when the evaluation mode is an examination activity mode.

Moreover, the camera may include a first camera capable of obtaining 3D information through a posture estimation algorithm, and a second camera capable of providing depth information.

Furthermore, the processor may obtain 3D body coordinate information based on the 3D information obtained by applying the posture estimation algorithm to an image captured through the first camera, and may obtain 3D body coordinate information by integrating 2D body coordinate information, which is obtained by applying the posture estimation algorithm to an image captured through the second camera, with depth information obtained through the second camera.

Also, the first image may include a specific posture or a specific activity related to evaluation among daily activities of the subject. The specific posture or the specific activity may be determined by an activity classifier. The second image may be an image captured as the subject performs a predetermined examination activity on the specific posture or the specific activity.

In addition, the processor may calculate an evaluation index for evaluating at least one of a spine and a joint associated with a corresponding activity based on a motion change at a point in time, when the subject takes the specific posture or the specific activity in the first image, and body coordinate information changed according to the motion change, and may determine whether at least one of the spine and the joint worsens or is improved, by comparing the calculated evaluation index with a previous evaluation index.

Besides, the processor may calculate an evaluation index for evaluating at least one of a spine and a joint associated with a corresponding activity based on a motion change at a point in time, when the subject takes the examination activity in the second image, and body coordinate information changed according to the motion change, and may determine whether at least one of the spine and the joint worsens or is improved, by comparing the calculated evaluation index with a previous evaluation index.

Moreover, the evaluation index may be set differently depending on a mechanism and an aspect of degeneration and a disease for each spine or each joint that is an evaluation target.

Furthermore, the evaluation index at a point in time when the evaluation target is a shoulder joint may include an index for evaluating a range of motion of the shoulder joint and an index for a workspace that quantifies a movement of a shoulder complex in three dimensions. The evaluation index at a point in time when the evaluation target is a lumbar spine may include an index for evaluating a sagittal vertical axis of the lumbar spine, a lumbar lordosis angle, and scoliosis on a coronal plane. The evaluation index at a point in time when the evaluation target is a cervical spine may include an index for evaluating a cervical lordosis angle, a chin-brow vertical axis (CBVA), and a sagittal vertical axis of the cervical spine. The evaluation index at a point in time when the evaluation target is a knee joint may include an index for evaluating a degree of knee varus/valgus deformity and a flexion contracture angle. The evaluation index at a point in time when the evaluation target is an ankle joint may include an index for evaluating a range of motion and a dynamic balance of the ankle joint.

Also, a cloud linked to a communication unit of the apparatus may store a format of at least one of the entire first image, the first image of which a resolution is changed to a low resolution, the first image with a lowered frame per second (fps), and skeleton information of the first image.

In addition, according to an aspect of the present disclosure for achieving the above-described technical task, a method performed by an apparatus using an image and an algorithm includes obtaining an image captured by a camera, calculating an evaluation index for evaluating at least one of a spine and a joint of a subject based on body coordinate information obtained from the image, and determining a state of at least one of the spine and the joint based on the calculated evaluation index. The image includes a first image, which is obtained when an evaluation mode is a routine activity mode, and a second image obtained when the evaluation mode is an examination activity mode.

Moreover, the camera may include a first camera capable of obtaining 3D information through a posture estimation algorithm, and a second camera capable of providing depth information.

Furthermore, the obtaining of the image may include obtaining 3D body coordinate information based on the 3D information obtained by applying the posture estimation algorithm to an image captured through the first camera, and obtaining 3D body coordinate information by integrating 2D body coordinate information, which is obtained by applying the posture estimation algorithm to an image captured through the second camera, with depth information obtained through the second camera.

Also, the first image may include a specific posture or a specific activity related to evaluation among daily activities of the subject. The specific posture or the specific activity may be determined by an activity classifier. The second image may be an image captured as the subject performs a predetermined examination activity on the specific posture or the specific activity.

In addition, the calculating of the evaluation index may include calculating an evaluation index for evaluating at least one of a spine and a joint associated with a corresponding activity based on a motion change at a point in time, when the subject takes the specific posture or the specific activity in the first image, and body coordinate information changed according to the motion change. The determining of the state may include determining whether at least one of the spine and the joint worsens or is improved, by comparing the calculated evaluation index with a previous evaluation index.

Besides, the calculating of the evaluation index may include calculating an evaluation index for evaluating at least one of a spine and a joint associated with a corresponding activity based on a motion change at a point in time, when the subject takes the examination activity in the second image, and body coordinate information changed according to the motion change. The determining of the state may include determining whether at least one of the spine and the joint worsens or is improved, by comparing the calculated evaluation index with a previous evaluation index.

Moreover, the evaluation index may be set differently depending on a mechanism and an aspect of degeneration and a disease for each spine or each joint that is an evaluation target.

Furthermore, the evaluation index at a point in time when the evaluation target is a shoulder joint may include an index for evaluating a range of motion of the shoulder joint and an index for a workspace that quantifies a movement of a shoulder complex in three dimensions. The evaluation index at a point in time when the evaluation target is a lumbar spine may include an index for evaluating a sagittal vertical axis of the lumbar spine, a lumbar lordosis angle, and scoliosis on a coronal plane. The evaluation index at a point in time when the evaluation target is a cervical spine may include an index for evaluating a cervical lordosis angle, a chin-brow vertical axis (CBVA), and a sagittal vertical axis of the cervical spine. The evaluation index at a point in time when the evaluation target is a knee joint may include an index for evaluating a degree of knee varus/valgus deformity and a flexion contracture angle. The evaluation index at a point in time when the evaluation target is an ankle joint may include an index for evaluating a range of motion and a dynamic balance of the ankle joint.

Also, a cloud linked to a communication unit of the apparatus may store a format of at least one of the entire first image, the first image of which a resolution is changed to a low resolution, the first image with a lowered frame per second (fps), and skeleton information of the first image.

Moreover, according to an aspect of the present disclosure for achieving the above-described technical task, a computer program, when executed by one or more processors, performs operations for performing a method performed by an apparatus using an image and an algorithm as the computer program stored in a computer-readable storage medium. The operations includes obtaining an image captured by a camera, calculating an evaluation index for evaluating at least one of a spine and a joint of a subject based on body coordinate information obtained from the image, and determining a state of at least one of the spine and the joint based on the calculated evaluation index. The image includes a first image, which is obtained when an evaluation mode is a routine activity mode, and a second image obtained when the evaluation mode is an examination activity mode.

In addition, a computer-readable recording medium for recording a computer program for performing the method for implementing the present disclosure may be further provided.

### Effect of Invention

According to the above-mentioned problem solving means of the present disclosure, non-intrusive analysis is possible without the help of experts and is not limited by the limitations of cost, equipment, or the situation being evaluated.

Moreover, spinal/joint deformities or diseases may be detected and treated before they become severe, and may be utilized for rehabilitation of spinal/joint diseases to improve treatment efficiency. This improves clinical outcomes and reduces costs.

In addition, accurate and helpful evaluation tools may be provided under the condition of limited access to healthcare, thereby improving the efficiency of healthcare resources and reducing unnecessary social costs. In particular, degenerative spinal/joint diseases are more prevalent in the elderly. Accordingly, the rapidly increasing medical costs associated with an aging population may be reduced by utilizing the evaluation tools.

Furthermore, because the evaluation is possible in the condition that access to healthcare is limited, the evaluation tool may also be applied to telemedicine or smart healthcare.

Effects of the present disclosure are not limited to the effects mentioned above, and other effects not mentioned will be apparent by those skilled in the art from the following description.

### DESCRIPTION OF DRAWINGS

FIG. 1 is a drawing schematically illustrating a musculoskeletal evaluation system using an image and an algorithm, according to an embodiment of the present disclosure.

FIG. 2 is a flowchart of a musculoskeletal evaluation method using an image and an algorithm, according to an embodiment of the present disclosure.

FIG. 3 is a drawing for describing the creation of a first image from an image obtained by capturing a routine activity, according to an embodiment of the present disclosure.

FIG. 4 is a drawing for describing generating a second image from an image obtained by capturing an examination activity, according to an embodiment of the present disclosure.

FIG. 5 is a diagram for describing obtaining body coordinate information through a first camera, according to an embodiment of the present disclosure.

FIG. 6 is a drawing for describing obtaining body coordinate information through a second camera, according to an embodiment of the present disclosure.

FIG. 7 is a diagram schematically illustrating a musculoskeletal evaluation system including a cloud, according to an embodiment of the present disclosure.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components. Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above. Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

Prior to a description, the meaning of terms used in the present specification will be described briefly. However, because the description of terms is used to help the understanding of this specification, it should be noted that when the present disclosure is not explicitly described as a limiting matter, it is not used in the sense of limiting the technical idea of the present disclosure.

In this specification, an ‘apparatus’ includes all various devices capable of providing results to a user by performing arithmetic processing. For example, the apparatus may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet (WiBro) terminal, and a wearable device such as a timepiece, a ring, a bracelet, an anklet, a necklace, glasses, a contact lens, or a head-mounted device (HMD).

Functions related to artificial intelligence according to an embodiment of the present disclosure are operated through a processor and a memory. The processor may consist of one or more processors. In this case, the one or more processors may be a general-purpose processor (e.g., a CPU, an AP, or a digital signal processor (DSP)), a graphics-dedicated processor (e.g., a GPU or a vision processing unit (VPU)), or an artificial intelligence (AI)-dedicated processor (e.g., an NPU). Under control of the one or more processors, input data may be processed depending on an AI model, or a predefined operating rule stored in the memory. Alternatively, when the one or more processors are AI-dedicated processors, the AI-dedicated processor may be designed with a hardware structure specialized for processing a specific AI model.

The predefined operating rule or the artificial intelligence model is created through learning. Here, being created through learning means creating the predefined operating rule or the artificial intelligence model configured to perform desired features (or purposes) as a basic artificial intelligence model is learned by using pieces of learning data by a learning algorithm. This learning may be performed by a device itself, on which the artificial intelligence according to an embodiment of the present disclosure is performed, or may be performed through a separate server and/or system. For example, the learning algorithm may include supervised learning, unsupervised learning, semi-supervised learning, or reinforcement learning, but may not be limited to the above example.

An artificial intelligence model may be composed of a plurality of neural network layers. The plurality of neural network layers respectively have a plurality of weight values, and each of the plurality of neural network layers performs neural network calculation through calculations between the calculation result of the previous layer and the plurality of weight values. The plurality of weight values of the plurality of neural network layers may be optimized by the learning result of the artificial intelligence model. For example, during a learning process, the plurality of weight values may be updated such that a loss value or a cost value obtained from the artificial intelligence model is reduced or minimized. The artificial neural network may include a deep neural network (DNN). The artificial neural network may be, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the above-described example.

The processor may create a neural network, may train or learn a neural network, or may perform operations based on received input data, and then may generate an information signal or may retrain the neural network based on the performed results.

It will be understood by those skilled in the art that a neural network may include any neural network, but is not limited to a convolutional neural network (CNN), a recurrent neural network (RNN), a perceptron, a multilayer perceptron, a feed forward (FF), a radial basis network (RBF), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN).

According to an embodiment of the present disclosure, the processor may use various artificial intelligence structures and algorithms such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) Network, a classification network, Generative Modeling, explainable AI, Continual AI, Representation Learning, AI for Material Design, algorithms for natural language processing (e.g., BERT, SP-BERT, MRC/QA, Text Analysis, Dialog System, GPT-3, and GPT-4), algorithms for vision processing (e.g., Visual Analytics, Visual Understanding, Video Synthesis, and ResNet), algorithms for data intelligence (e.g., Anomaly Detection, Prediction, Time-Series Forecasting, Optimization, Recommendation, and Data Creation), but is not limited thereto.

Hereinafter, an embodiment of the present disclosure will be described in detail with reference to the accompanying drawings.

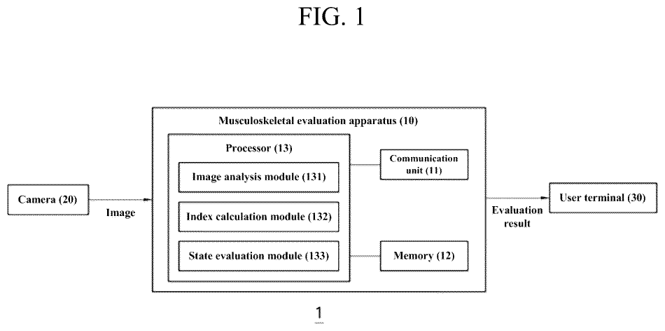

FIG. 1 is a drawing schematically illustrating a musculoskeletal evaluation system 1 (hereinafter, referred to as a “system”) using an image and an algorithm, according to an embodiment of the present disclosure.

According to an embodiment of the present disclosure, the system 1 may include a musculoskeletal evaluation apparatus 10, a camera 20, and a user terminal 30. However, in some embodiments, the system 1 may include fewer or more components than the components illustrated in FIG. 1.

The musculoskeletal evaluation apparatus 10 may receive an image captured by the camera 20, may process the received image to generate images (or consecutive images) including postures or movements significant for spine and joint evaluation, may calculate an evaluation index using body coordinate information about the generated images, and may evaluate health and disease states of spines and joints, or alignment states of spines and joints, by using the calculated evaluation index.

The musculoskeletal evaluation apparatus 10 may provide the evaluation results to the user terminal 30. In detail, the user terminal 30 may install an app or a program that provides a musculoskeletal evaluation service and may identify the evaluation results within the app.

Here, the user terminal 30 may be a subject's terminal or a medical personnel's terminal. The subject may identify and manage the condition of his/her spines and joints on his/her own without the help of a professional through the evaluation results. The medical personnel may diagnose the subject's spine and joint deformities early through the evaluation results and may identify objective information necessary to observe the progression.

An information processing means such as a computer may be applied to the user terminal 30. The user terminal 30 may include a processor such as a control unit, a photographing means such as a camera, an input/output means including a touch screen, and may mean any device including a communication function. That is, it may be applied to any device such as a smartphone, a tablet, a PDA, a laptop, or a desktop.

In an embodiment, the user terminal 30 may perform the operations of the musculoskeletal evaluation apparatus 10 in an on-device format. In detail, the user terminal 30 may process images captured by the camera 20 through the app or program installed on the user terminal 30 to generate images (or consecutive images) including postures or movements significant for spine and joint evaluation, may calculate an evaluation index using body coordinate information about the generated images, and may evaluate health and disease states of spines and joints, or alignment states of spines and joints, by using the calculated evaluation index.

The camera 20 may include a first camera, such as an RGB camera, and a second camera, such as a depth camera, that provides 3D information.

In detail, the first camera may be a camera capable of obtaining two-dimensional (2D) or three-dimensional (3D) information through a pose estimation algorithm, and may refer to a general video camera or a smartphone camera. That is, the first camera may not directly provide 2D or 3D information, and the 2D or 3D information may be obtained by applying a pose estimation algorithm to the image captured by the first camera.

The second camera may be a depth camera, a camera that calculates depth by using an infrared emitter/sensor, or a camera on a smartphone that includes a LiDAR sensor. However, an embodiment is not limited thereto, and any camera capable of providing 3D information may be applied thereto.

Referring to FIG. 1, the musculoskeletal evaluation apparatus 10 may include a communication unit 11, a memory 12, and a processor 13. The processor 13 may include an image analysis module 131, an index calculation module 132, and a state evaluation module 133. However, in some embodiments, each of the musculoskeletal evaluation apparatus 10 and the processor 13 may include fewer or more components than the components illustrated in FIG. 1.

The communication unit 11 may include one or more modules that enable wireless or wired communication between the musculoskeletal evaluation apparatus 10 and the camera 20, between the musculoskeletal evaluation apparatus 10 and the user terminal 30, between the musculoskeletal evaluation apparatus 10 and an external device (not shown), or between the musculoskeletal evaluation apparatus 10 and a communication network. For example, the communication unit 11 may include at least one of a wired communication module, a wireless communication module, a short-range communication module, and a location information module.

Various types of communication networks may be used. For example, wireless communication methods such as wireless LAN (WLAN), Wi-Fi, Wibro, Wimax, High Speed Downlink Packet Access (HSDPA), and the like or wired communication methods such as Ethernet, xDSL (ADSL or VDSL), Hybrid Fiber Coax (HFC), Fiber to The Curb (FTTC), Fiber To The Home (FTTH), and the like may be used in a communication network.

In the meantime, the communication network is not limited to the communication method described above, and may include all types of communication methods widely known or to be developed in the future in addition to the above communication methods.

Here, in addition to various wired communication modules such as a Local Area Network (LAN) module, a Wide Area Network (WAN) module, or a Value Added Network (VAN) module, the wired communication module may include a variety of cable communication modules such as Universal Serial Bus (USB), High Definition Multimedia Interface (HDMI), Digital Visual Interface (DVI), recommended standard232 (RS-232), power line communication, or plain old telephone service (POTS).

Here, the wireless communication module may include a wireless communication module for supporting various wireless communication methods such as Global System for Mobile (GSM) communication, Code Division Multiple Access (CDMA), Wideband Code Division Multiple Access (WCDMA), Universal Mobile Telecommunication System (UMTS), Time Division Multiple Access (TDMA), Long Term Evolution (LTE), 4G, 5G, and 6G in addition to a Wi-Fi module and Wireless broadband module.

The short-range communication module may be used for short range communication, and may support short-range communication by using at least one of Bluetooth™, radio frequency identification (RFID), infrared data association (IrDA), ultra wideband (UWB), ZigBee, near field communication (NFC), Wireless-Fidelity (Wi-Fi), Wi-Fi Direct, and wireless universal serial bus (Wireless USB) technologies.

The memory 12 may store at least one process for performing musculoskeletal evaluation by using an image and an algorithm.

The memory 12 may store data supporting various functions of the musculoskeletal evaluation apparatus 10 and a program for the operation of the processor 13, may store pieces of input/output data (e.g., music files, still images, videos, and the like), and may store a plurality of application programs (or applications) running on the musculoskeletal evaluation apparatus 10, data for operations of the musculoskeletal evaluation apparatus 10, and commands. At least part of the application programs may be downloaded from an external server through wireless communication.

The memory 12 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 12 may be a database that is separate from the musculoskeletal evaluation apparatus 10, but is connected by wire or wirelessly.

The processor 13 may execute the above-described operations by using a memory that stores data regarding an algorithm for controlling operations of components within the musculoskeletal evaluation apparatus 10, or a program for implementing the algorithm, and data stored in the memory. In this case, each of the memory 12 and the processor 13 may be implemented as separate chips. Alternatively, the memory 12 and the processor 13 may be implemented as a single chip.

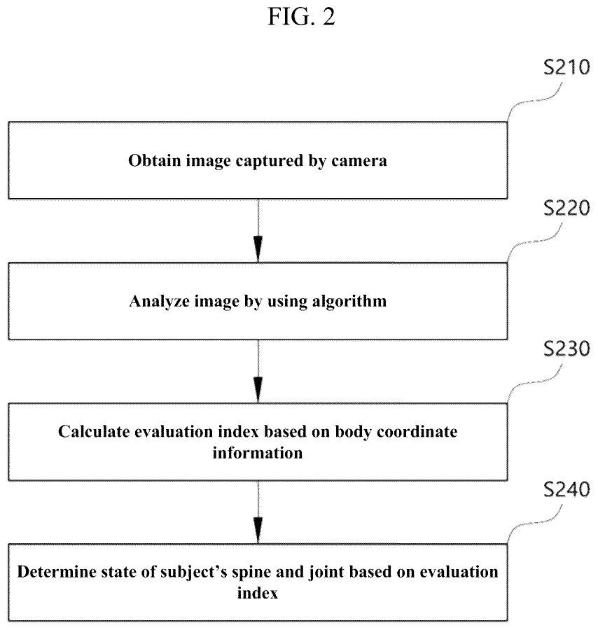

Furthermore, the processor 13 may control one of the components described above or the combination of the components to implement various embodiments of the present disclosure described below with reference to FIGS. 2 to 6 on the musculoskeletal evaluation apparatus 10.

The image analysis module 131 of the processor 13 may analyze an image (video) captured by the camera 20 by using at least one of a personal identification algorithm and a pose estimation algorithm.

The index calculation module 132 of the processor 13 may calculate an evaluation index for evaluating at least one of a spine and a joint based on the body coordinate information obtained through analysis.

The state evaluation module 133 of the processor 13 may determine the state of at least one of the subject's spine and joint based on the calculated evaluation index.

Hereinafter, a first embodiment of a musculoskeletal evaluation method using an image and an algorithm will be described in detail with reference to FIGS. 2 to 6.

FIG. 2 is a flowchart of a musculoskeletal evaluation method using an image and an algorithm, according to an embodiment of the present disclosure.

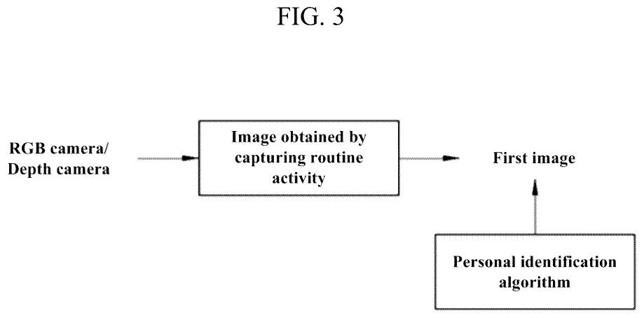

FIG. 3 is a drawing for describing the creation of a first image from an image obtained by capturing a routine activity, according to an embodiment of the present disclosure.

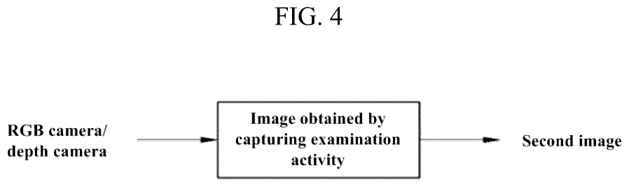

FIG. 4 is a drawing for describing generating a second image from an image obtained by capturing an examination activity, according to an embodiment of the present disclosure.

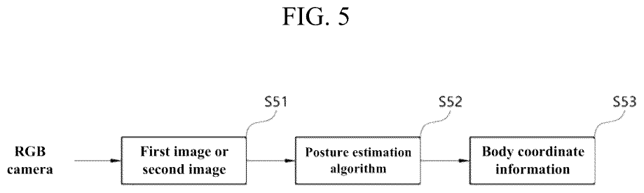

FIG. 5 is a diagram for describing obtaining body coordinate information through a first camera, according to an embodiment of the present disclosure.

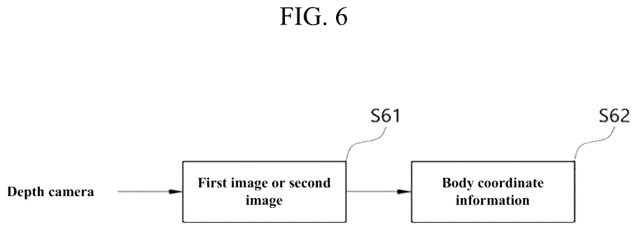

FIG. 6 is a drawing for describing obtaining body coordinate information through a second camera, according to an embodiment of the present disclosure.

For convenience of description, below, each of steps is described as being performed by the processor 13, but each step may be understood as being performed by any one of the image analysis module 131, the index calculation module 132, the state evaluation module 133, and modules not shown in FIG. 1 included in the processor 13.

Referring to FIG. 2, the processor 13 of the musculoskeletal evaluation apparatus 10 may obtain an image captured by the camera 20 (S210).

As described above, the camera 20 may include a first camera, such as an RGB camera, and a second camera, such as a depth camera, that provides 3D information.

The image captured by the camera 20 (a first camera or a second camera) may include a first image for a routine activity and a second image for an examination activity.

Here, the first image may be image data obtained when an evaluation mode is a routine activity mode, and the second image may be image data acquired when the evaluation mode is an examination activity mode.

The processor 13 may utilize the first image to extract information meaningful for evaluation from the subject's daily life activities, or may utilize the second image to obtain accurate information as the subject consciously performs examination activities.

In detail, the first image may be image data obtained by capturing the subject's routine activity. The first image may include a specific posture or a specific activity related to the evaluation among the subject's daily activities.

Here, the posture may mean a static body position state (e.g., a standing posture). The activity may mean a dynamic body movement (e.g., walking).

The first image obtained by capturing the subject's daily activities may be stored and archived in a cloud linked to the communication unit. In this case, the entire first image may be stored in the cloud; the first image may be stored with a lower resolution or lower frames per second (fps); or, only skeleton information of the first image may be stored.

In other words, according to an embodiment, the first image may be stored in the cloud. The first image may be stored in the form of at least one of the entire first image, the first image of which the resolution is changed to a low resolution, the first image with a lowered fps, and the skeleton information of the first image.

Besides, the first image stored in the cloud may be volatilized after a certain period of time for privacy reasons.

In an embodiment, the processor 13 may provide the cloud with the moving part of the first image. Alternatively, the processor 13 may provide the cloud with time information about the moving part of the first image together with the entire first image.

According to an embodiment, when a plurality of objects (people) appear in the first image, the processor 13 may recognize the subject through pre-stored identification information and may provide the cloud with only a portion where the subject appears. Alternatively, the processor 13 may provide the cloud with time information about a portion of the first image where the subject appears, together with the entire first image.

An activity classifier may determine a specific posture or a specific activity included in the first image stored in the cloud. The activity classifier may store, in advance, posture or activity information to be determined, and thus may detect postures or activities which are meaningful for evaluation and which are included in the first image.

According to an embodiment, the posture or activity information stored in the activity classifier may be updated. In detail, additional posture or activity information may be updated to evaluate an existing disease, or the posture or activity information may be updated to evaluate a new disease.

The processor 13 may analyze whether the subject's joint and/or spine is aligned and deformed, through a specific posture or a specific activity determined by the activity classifier.

For example, when an upright posture is detected from the first image by the activity classifier, the processor 13 may analyze the alignment of the spine on a sagittal plane or the deformity of the knees on the coronal plane, through the subject's upright posture.

For another example, when an arm-raising activity is detected from the first image by the activity classifier, the processor 13 may analyze the subject's shoulder joint movements (movement patterns and the range of motion) based on the arm-raising activity.

For another example, when a sitting posture is detected from the first image by the activity classifier, the processor 13 may analyze the deformation of the subject's cervical and thoracic spine through the subject's sitting posture.

For another example, when a sitting posture is detected from the first image by the activity classifier, the processor 13 may analyze the subject's knee joint motion (movement patterns and the range of motion) on the sagittal plane through the sitting activity.

As another example, when a walking activity is detected from the first image by the activity classifier, the processor 13 may analyze the movements (movement patterns and the range of motion) of the subject's spine, knee joints, and ankle joints through the subject's walking activity.

Moreover, the second image may be an image generated as the subject performs a predetermined examination activity on a specific posture or a specific activity related to the evaluation. Here, the examination activity may be set to a posture or an activity associated with the evaluation target.

The second image obtained by capturing the subject's examination activities may be stored and archived in a cloud linked to the communication unit. In this case, the entire second image may be stored in the cloud; the second image may be stored with a lower resolution or lower frames per second (fps); or, only skeleton information of the second image may be stored.

In other words, according to an embodiment, the second image may be stored in the cloud. The second image may be stored in the form of at least one of the entire second image, the second image of which the resolution is changed to a low resolution, the second image with a lowered fps, and the skeleton information of the second image.

Besides, the second image stored in the cloud may be volatilized after a certain period of time for privacy reasons.

For example, when the evaluation target is a spine and a joint, the examination activity may be set to maintain an upright posture on a front view or a side view for a predetermined period of time. The processor 13 may analyze the alignment of the subject's spine on the sagittal plane or the deformation of the knee on the coronal plane through the second image obtained by capturing the subject's upright posture.

For another example, when the evaluation target is a shoulder joint, the examination activity may be set to perform activities necessary for functional evaluation of the shoulder joint. The processor 13 may analyze the range of motion and a movement pattern of the subject's shoulder joint through the second image obtained by capturing the examination activity.

For another example, when the evaluation target is a knee joint, the examination activity may be set to perform activities necessary for evaluating the function of the knee joint. The processor 13 may analyze the range of motion and a movement pattern of the subject's knee joint through the second image obtained by capturing the examination activity.

For another example, when the evaluation target is a spine, the examination activity may be set to perform forward or backward bending activities necessary for functional evaluation of the spine. The processor 13 may analyze the range of motion and a movement pattern of the subject's spine through the second image obtained by capturing the examination activity.

The processor 13 of the musculoskeletal evaluation apparatus 10 may analyze the image by using at least one of a personal identification algorithm and a posture estimation algorithm (S220).

The processor 13 may determine whether to apply the personal identification algorithm, depending on an evaluation mode. The personal identification algorithm may be applied only when the evaluation mode is a daily activity mode.

That is, because the first image obtained by capturing the subject's daily life may be difficult to be identified (as described above, when there are a plurality of objects in the first image.), a process for identifying the subject may be necessary. Accordingly, the processor 13 may identify the subject in the first image by using the personal identification algorithm.

In detail, the processor 13 may identify the subject in the first image based on the identification information (e.g., a facial image) entered by the subject when the subject registers a musculoskeletal evaluation service.

When a face detected from the first image is identified as a user registered with the service, the processor 13 may provide and output the first image to the cloud for storage.

When the face detected from the first image is determined not to correspond to a user registered with the service, the processor 13 may not provide the first image to the cloud.

Referring to FIG. 3, the first image may be created by capturing a routine activity through an RGB camera or a depth camera. Whether the object detected from the first image is the subject may be determined by applying a personal identification algorithm to the generated first image.

According to an embodiment, when a subject is detected from the first image through the personal identification algorithm, the processor 13 may provide the cloud with only the portion of the first image where the subject is moving. Alternatively, the processor 13 may provide the cloud with time information about the portion of the first image, where the subject moves, together with the entire first image.

According to an embodiment, when the subject among a plurality of objects is detected through a personal identification algorithm when a plurality of objects (people) appear in the first image, the processor 13 may provide the cloud with only the portion where the subject appears. Alternatively, the processor 13 may provide the cloud with time information about a portion of the first image where the subject appears, together with the entire first image.

As described above, it is described that a personal identification algorithm is used for object identification, but it is not limited thereto. A facial recognition or gait pattern recognition algorithm may also be used.

On the other hand, there is no need to apply a personal identification algorithm to the second image obtained as the subject performs an examination activity, because the subject to be captured is predetermined.

Referring to FIG. 4, when an image obtained by capturing the examination activity is obtained through an RGB camera or depth camera, the corresponding image may be generated as the second image.

Additionally, the processor 13 may determine whether to apply the posture estimation algorithm, depending on whether the camera is the first camera or the second camera.

That is, the processor 13 may obtain 2D or 3D information by applying a machine learning-based posture estimation algorithm to the image captured through the first camera, and may obtain body coordinate information based thereon.

Referring to FIG. 5, when processing/analyzing an image captured through the RGB camera (the first camera) to obtain a first image or a second image (S51), the processor 13 may apply the posture estimation algorithm to the first image or the second image (S52) and may obtain the body coordinate information about the first image or the second image (S53).

On the other hand, the processor 13 may immediately obtain the body coordinate information without a need to apply a machine learning-based posture estimation algorithm to the image captured by the second camera capable of providing depth information.

Referring to FIG. 6, when processing/analyzing the image captured by the depth camera (the second camera) to obtain a first image or a second image (S61), the processor 13 may immediately obtain 3D body coordinate information about the first image or the second image (S62).

Although FIG. 6 illustrates that the 3D body coordinate information is capable of being immediately obtained without applying the posture estimation algorithm to the image captured through the depth camera, but it is not limited thereto. According to an embodiment, the posture estimation algorithm may also be applied to the image captured through the depth camera. In detail, the 3D body coordinate information may be extracted by integrating the depth information, which is obtained through the depth camera, with the 2D body coordinate information obtained by applying a posture estimation algorithm to the image captured through the depth camera.

In operation S51 and operation S61, the first image may be generated through the operation described with reference to FIG. 3 when the image captured through the camera is a routine activity, and the second image may be generated through the operation described with reference to FIG. 4 when the image captured through the camera is an examination activity.

The processor 13 of the musculoskeletal evaluation apparatus 10 may calculate an evaluation index for evaluating at least one of a spine and a joint based on the body coordinate information obtained through analysis (S230).

Here, the evaluation index may be set differently depending on the mechanism and aspect of degeneration and disease for each spine or each joint that is an evaluation target.

For example, when the evaluation target is a shoulder joint, it is important to evaluate the kinematics of a shoulder complex, and thus an index for evaluating the range of motion of the shoulder joint may be set primarily. Moreover, an index for a workspace that quantifies the movement of the shoulder complex in three dimensions in situations where a specific activity is performed may be set.

When the evaluation target is a lumbar spine, an index may be set to evaluate a sagittal vertical axis, a lumbar lordosis angle, and scoliosis on the coronal plane. When the evaluation target is a cervical spine, a cervical lordosis angle, a chin-brow vertical axis (CBVA), and a cervical sagittal vertical axis may be important, and thus an index may be set to evaluate them.

When the evaluation target is a knee joint, the degree of knee varus/valgus deformity and the flexion contracture angle are important, and thus an index may be set to evaluate them. Moreover, an index for lower limb strength may be set by analyzing information about a knee joint movement within the subject's specific activity.

When the evaluation target is an ankle joint, the range of motion and the dynamic balance of the ankle joint may be important, and thus an index may be set to evaluate them.

In detail, the processor 13 may determine the type of evaluation for a specific posture or a specific activity included in an image, and may calculate an evaluation index for a spine or a joint associated with the corresponding evaluation posture or the corresponding evaluation activity based on body coordinate information about the corresponding image.

According to an embodiment, the processor 13 may calculate an evaluation index for evaluating at least one of a spine and a joint associated with the corresponding posture or the corresponding activity based on body coordinate information and a movement change at a point in time when the subject takes a specific posture or a specific activity in the first image.

According to an embodiment, the processor 13 may calculate an evaluation index for evaluating at least one of a spine and a joint associated with the corresponding activity based on a motion change at a point in time, when the subject takes a specific examination activity in the second image, and the body coordinate information. As described above, the examination activity may be set to a posture or an activity associated with the evaluation target.

For example, in the case of a shoulder joint, as described above, because an index for evaluating the range of motion of the shoulder joint, and an index for a workspace that quantifies the movement of the shoulder complex in three dimensions may be set as an evaluation index, the processor 13 may calculate an actual value (e.g., the actual range of motion) for the evaluation index of the subject's shoulder joint based on body coordinate information changed depending on movement changes in a posture or activity associated with the shoulder joint during the subject's routine activities or movement changes in examination activities of the shoulder joint.

In the case of a lumbar spine, as described above, because an index for evaluating a sagittal vertical axis, a lumbar lordosis angle, and scoliosis on a coronal plane may be set as an evaluation index, the processor 13 may calculate an actual value (e.g., an actual lumbar lordosis angle) for the evaluation index of the subject's lumbar spine based on body coordinate information changed depending on movement changes in a posture or activity associated with the lumbar spine during the subject's routine activities or movement changes in examination activities of the lumbar spine.

In the case of a cervical spine, as described above, because an index for evaluating a cervical lordosis angle, a chin-brow vertical axis (CBVA), and a cervical sagittal vertical axis may be set as an evaluation index, the processor 13 may calculate an actual value (e.g., the actual cervical lordosis angle) for the evaluation index of the subject's cervical spine based on body coordinate information changed depending on movement changes in a posture or activity associated with the cervical spine during the subject's routine activities or movement changes in examination activities of the lumbar spine.

In the case of a knee joint, as described above, because an index for evaluating the degree of knee varus/valgus deformity and the flexion contracture angle may be set as an evaluation index, the processor 13 may calculate an actual value (e.g., the actual degree of knee varus/valgus deformity) for the evaluation index of the subject's knee joint based on body coordinate information changed depending on movement changes in a posture or activity associated with the knee joint during the subject's routine activities or movement changes in examination activities of the lumbar spine.

In the case of an ankle joint, as described above, because an index for evaluating the range of motion and the dynamic balance of the ankle joint may be set as an evaluation index, the processor 13 may calculate an actual value (e.g., the actual range of motion of the ankle joint) for the evaluation index of the subject's ankle joint based on body coordinate information changed depending on movement changes in a posture or activity associated with the ankle joint during the subject's routine activities or movement changes in examination activities of the lumbar spine.

The processor 13 of the musculoskeletal evaluation apparatus 10 may determine the state of at least one of the subject's spine and joint based on the calculated evaluation index (S240).

The processor 13 may quantitatively evaluate and report the degree of joint degeneration (aging) through the calculated evaluation index.

According to an embodiment, the processor 13 may determine whether at least one of the spine and the joint worsens or is improved, by comparing the calculated evaluation index with the previous evaluation index.

For example, when the currently calculated actual range of motion of the shoulder joint is smaller than the previously calculated actual range of motion of the shoulder joint, in the case where the actual range of motion of the shoulder joint is used as an evaluation index when a shoulder joint is evaluated, the subject's shoulder joint state may be determined to have worsened compared to before.

According to an embodiment, the processor 13 may determine whether the evaluation target corresponds to a pathological state, based on the calculated evaluation index.

For example, in the case where the lordosis angle of the lumbar spine is used as an evaluation index during lumbar spine evaluation, when the actual lordosis angle of the currently calculated lumbar spine is outside a predetermined reference range, the subject's lumbar spine may be determined to be in a pathological state. Here, the reference range may be set for each subject based on the subject's characteristic information, such as the subject's age, whether the subject smokes, the subject's weight, previous evaluation results, and related diseases.

The processor 13 may provide a rehabilitation method for an abnormal spine and/or an abnormal joint or may suggest a visit to a medical institution based on these evaluation results.

According to an embodiment, when there are a plurality of evaluation indices for a spine or a joint associated with one evaluation posture, the processor 13 may calculate an integrated evaluation score based on a predetermined weight for each of the plurality of evaluation indices. Here, the weight may be set differently depending on the extent to which it contributes to the accuracy of the evaluation results for each evaluation index.

For example, the upright posture may be used to evaluate states of a lumbar spine and a knee joint. In this case, as described above, the sagittal vertical axis of the lumbar spine, the lordosis angle of the lumbar spine, the degree of knee varus/valgus deformity, and a flexion contracture angle may be set as evaluation indices. In this case, among the four evaluation indices, a higher weight may be given in order depending on the extent to which it contributes to the evaluation accuracy.

The processor 13 may calculate an integrated evaluation score for the state of the subject's lumbar spine and knee joint by differently applying weights to the sagittal vertical axis of the lumbar spine, the lordosis angle of the lumbar spine, the degree of knee varus/valgus deformity of the knee joint, and a flexion contracture angle, which are calculated based on body coordinate information.

When the calculated integrated evaluation score is less than a predetermined reference score, the processor 13 may determine the abnormality (pathological state) for the evaluation target.

In this case, the processor 13 may re-calculate each of the plurality of evaluation indices without applying weights, may compare the calculated value with each predetermined reference range, and may determine that an evaluation index falling outside a reference range is the factor of anomaly occurrence. The processor 13 may provide a training method for the evaluation index determined to be the factor of anomaly occurrence.

In detail, when the integrated evaluation score for the subject's lumbar spine state and knee joint state is less than a predetermined reference score, the processor 13 may compare the reference range with the evaluation indices (e.g., the sagittal vertical axis of the lumbar spine, the lordosis angle of the lumbar spine, the degree of knee varus/valgus deformity, and the flexion contracture angle). When one (e.g., the lordosis angle of the lumbar spine) of the four evaluation indices falls outside the reference range, the processor 13 may determine the lordosis angle of the lumbar spine as the factor of the anomaly occurrence and may provide the subject with an intensive training method for the corresponding evaluation index.

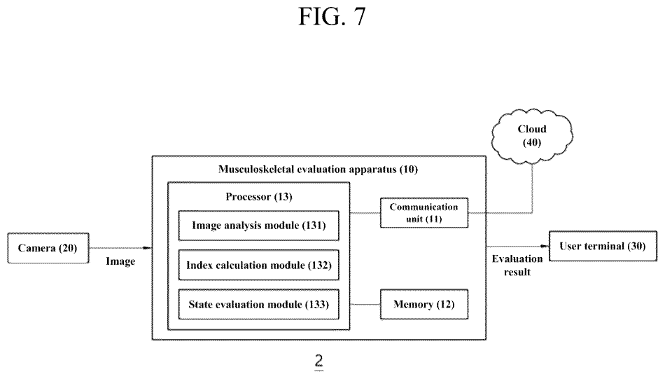

Hereinafter, a system including a cloud in the system described in FIG. 1 will be described with reference to FIG. 7.

FIG. 7 is a diagram schematically illustrating a musculoskeletal evaluation system 2 including a cloud, according to an embodiment of the present disclosure.

Referring to FIG. 7, the system 2 may include the musculoskeletal evaluation apparatus 10, the camera 20, the user terminal 30, and a cloud 40. However, in some embodiments, the system 2 may include fewer or more components than the components illustrated in FIG. 7.

The operations of the musculoskeletal evaluation apparatus 10, the camera 20, and the user terminal 30 illustrated in FIG. 7 are the same as those described with reference to FIGS. 1 to 6, and thus detailed descriptions thereof will be omitted.

The cloud 40 added in FIG. 7 may be linked to the communication unit 11 of the musculoskeletal evaluation apparatus 10 and may store and archive a first image or a second image transmitted from the communication unit 11.

The entire first image may be stored in the cloud 40; the first image may be stored with a lower resolution or lower frames per second (fps); or, only skeleton information of the first image may be stored.

In other words, according to an embodiment, the first image may be stored in the cloud 40. The first image may be stored in the form of at least one of the entire first image, the first image of which the resolution is changed to a low resolution, the first image with a lowered fps, and the skeleton information of the first image.

Besides, the first image stored in the cloud may be volatilized after a certain period of time for privacy reasons.

As described above, only the storage method of the first image is described, but the second image may also be transmitted and stored in various forms in the same manner.

In an embodiment, the processor 13 may provide the cloud 40 with the moving part of the first image. Alternatively, the processor 13 may provide the cloud 40 with time information about the moving part of the first image together with the entire first image.

According to an embodiment, when a plurality of objects (people) appear in the first image, the processor 13 may recognize the subject through pre-stored identification information and may provide the cloud 40 with only a portion where the subject appears. Alternatively, the processor 13 may provide the cloud 40 with time information about a portion of the first image where the subject appears, together with the entire first image.

As described above, the cloud 40 may perform the role of storing and archiving images. According to an embodiment, the cloud 40 may perform an analysis/evaluation operation of the processor 13 described above and a detection/determination operation of an activity classifier.

FIG. 2 illustrates that steps are performed sequentially. However, this is merely illustrative of the technical idea of the present disclosure. Those skilled in the art to which an embodiment of the present disclosure belongs may apply various modifications and variations by changing and performing the order of steps illustrated in FIG. 2 or performing steps in parallel without departing from the essential characteristics of an embodiment of the present disclosure. The steps illustrated in FIG. 2 are not limited to a time-series order.

In the meantime, in the above description, steps described in FIG. 2 may be further divided into additional operations or may be combined into fewer steps, according to an embodiment of the present disclosure. In addition, some steps may be omitted as necessary, and the order between steps may be changed.

Meanwhile, the disclosed embodiments may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

1. A musculoskeletal evaluation apparatus using an image and an algorithm, the apparatus comprising:
a memory configured to store at least one process for performing musculoskeletal evaluation by using an image and an algorithm;
a processor configured to perform an operation according to the process, wherein the processor is configured to:
obtain an image captured by a camera;
calculate an evaluation index for evaluating at least one of a spine and a joint of a subject based on body coordinate information obtained from the image; and
determine a state of at least one of the spine and the joint based on the calculated evaluation index, and
wherein the image includes a first image, which is obtained when an evaluation mode is a routine activity mode, and a second image obtained when the evaluation mode is an examination activity mode.

2. The apparatus of claim 1, wherein the camera includes a first camera capable of obtaining three-dimensional (3D) information through a posture estimation algorithm, and a second camera capable of providing depth information.

3. The apparatus of claim 2, wherein the processor is configured to:
obtain 3D body coordinate information based on the 3D information obtained by applying the posture estimation algorithm to an image captured through the first camera; and
obtain 3D body coordinate information by integrating two-dimensional (2D) body coordinate information, which is obtained by applying the posture estimation algorithm to an image captured through the second camera, with depth information obtained through the second camera.

4. The apparatus of claim 1, wherein the first image includes a specific posture or a specific activity related to evaluation among daily activities of the subject,
wherein the specific posture or the specific activity is determined by an activity classifier, and
wherein the second image is an image captured as the subject performs a predetermined examination activity on the specific posture or the specific activity.

5. The apparatus of claim 4, wherein the processor is configured to:
calculate an evaluation index for evaluating at least one of a spine and a joint associated with a corresponding activity based on a motion change at a point in time, when the subject takes the specific posture or the specific activity in the first image, and body coordinate information changed according to the motion change; and
determine whether at least one of the spine and the joint worsens or is improved, by comparing the calculated evaluation index with a previous evaluation index.

6. The apparatus of claim 4, wherein the processor is configured to:
calculate an evaluation index for evaluating at least one of a spine and a joint associated with a corresponding activity based on a motion change at a point in time, when the subject takes the examination activity in the second image, and body coordinate information changed according to the motion change; and
determine whether at least one of the spine and the joint worsens or is improved, by comparing the calculated evaluation index with a previous evaluation index.

7. The apparatus of claim 1, wherein the evaluation index is set differently depending on a mechanism and an aspect of degeneration and a disease for each spine or each joint that is an evaluation target.

8. The apparatus of claim 7, wherein the evaluation index at a point in time when the evaluation target is a shoulder joint includes an index for evaluating a range of motion of the shoulder joint and an index for a workspace that quantifies a movement of a shoulder complex in three dimensions,
wherein the evaluation index at a point in time when the evaluation target is a lumbar spine includes an index for evaluating a sagittal vertical axis of the lumbar spine, a lumbar lordosis angle, and scoliosis on a coronal plane,
wherein the evaluation index at a point in time when the evaluation target is a cervical spine includes an index for evaluating a cervical lordosis angle, a chin-brow vertical axis (CBVA), and a sagittal vertical axis of the cervical spine,
wherein the evaluation index at a point in time when the evaluation target is a knee joint includes an index for evaluating a degree of knee varus/valgus deformity and a flexion contracture angle, and
wherein the evaluation index at a point in time when the evaluation target is an ankle joint includes an index for evaluating a range of motion and a dynamic balance of the ankle joint.

9. The apparatus of claim 1, wherein a cloud linked to a communication unit of the apparatus stores a format of at least one of the entire first image, the first image of which a resolution is changed to a low resolution, the first image with a lowered frame per second (fps), and skeleton information of the first image.

10. A method performed by an apparatus using an image and an algorithm, the method comprising:
obtaining an image captured by a camera;
calculating an evaluation index for evaluating at least one of a spine and a joint of a subject based on body coordinate information obtained from the image; and
determining a state of at least one of the spine and the joint based on the calculated evaluation index, and
wherein the image includes a first image, which is obtained when an evaluation mode is a routine activity mode, and a second image obtained when the evaluation mode is an examination activity mode.

11. The method of claim 10, wherein the camera includes a first camera capable of obtaining 3D information through a posture estimation algorithm, and a second camera capable of providing depth information.

12. The method of claim 11, wherein the obtaining of the image includes:
obtaining 3D body coordinate information based on the 3D information obtained by applying the posture estimation algorithm to an image captured through the first camera; and
obtaining 3D body coordinate information by integrating 2D body coordinate information, which is obtained by applying the posture estimation algorithm to an image captured through the second camera, with depth information obtained through the second camera.

13. The method of claim 10, wherein the first image includes a specific posture or a specific activity related to evaluation among daily activities of the subject,
wherein the specific posture or the specific activity is determined by an activity classifier, and
wherein the second image is an image captured as the subject performs a predetermined examination activity on the specific posture or the specific activity.

14. The method of claim 13, wherein the calculating of the evaluation index includes:
calculating an evaluation index for evaluating at least one of a spine and a joint associated with a corresponding activity based on a motion change at a point in time, when the subject takes the specific posture or the specific activity in the first image, and body coordinate information changed according to the motion change, and
wherein the determining of the state includes:
determining whether at least one of the spine and the joint worsens or is improved, by comparing the calculated evaluation index with a previous evaluation index.

15. The method of claim 13, wherein the calculating of the evaluation index includes:
calculating an evaluation index for evaluating at least one of a spine and a joint associated with a corresponding activity based on a motion change at a point in time, when the subject takes the examination activity in the second image, and body coordinate information changed according to the motion change, and
wherein the determining of the state includes:
determining whether at least one of the spine and the joint worsens or is improved, by comparing the calculated evaluation index with a previous evaluation index.

16. The method of claim 10, wherein the evaluation index is set differently depending on a mechanism and an aspect of degeneration and a disease for each spine or each joint that is an evaluation target.

17. The method of claim 16, wherein the evaluation index at a point in time when the evaluation target is a shoulder joint includes an index for evaluating a range of motion of the shoulder joint and an index for a workspace that quantifies a movement of a shoulder complex in three dimensions,
wherein the evaluation index at a point in time when the evaluation target is a lumbar spine includes an index for evaluating a sagittal vertical axis of the lumbar spine, a lumbar lordosis angle, and scoliosis on a coronal plane,
wherein the evaluation index at a point in time when the evaluation target is a cervical spine includes an index for evaluating a cervical lordosis angle, a CBVA, and a sagittal vertical axis of the cervical spine,
wherein the evaluation index at a point in time when the evaluation target is a knee joint includes an index for evaluating a degree of knee varus/valgus deformity and a flexion contracture angle, and
wherein the evaluation index at a point in time when the evaluation target is an ankle joint includes an index for evaluating a range of motion and a dynamic balance of the ankle joint.

18. The method of claim 10, wherein a cloud linked to a communication unit of the apparatus stores a format of at least one of the entire first image, the first image of which a resolution is changed to a low resolution, the first image with a lowered fps, and skeleton information of the first image.

19. A computer program, when executed by one or more processors, performing operations for performing a method performed by an apparatus using an image and an algorithm as the computer program stored in a computer-readable storage medium, the operations comprising:
obtaining an image captured by a camera;
calculating an evaluation index for evaluating at least one of a spine and a joint of a subject based on body coordinate information obtained from the image; and
determining a state of at least one of the spine and the joint based on the calculated evaluation index, and
wherein the image includes a first image, which is obtained when an evaluation mode is a routine activity mode, and a second image obtained when the evaluation mode is an examination activity mode.
